(19) United States
(12) Patent Application Publication
(10) Pub. No.: US 2010/0260738 A1
(43) Pub. Date: Oct. 14, 2010

(54) USES AND METHODS RELATING TO NDR KINASE EXPRESSION AND/OR ACTIVITY

(76) Inventors: Hauke Hermann Karl Cornils, Basel (CH); Stephan Dirnhofer, Basel (CH); Arthur Brian Hemmings, Bettingen (CH)

Correspondence Address: GIFFORD, KRASS, SPRINKLE,ANDERSON & CITKOWSKI, P.C, PO BOX 7021, TROY, MI, 48007-7021, US

(21) Appl. No.: 12/516,877

(22) PCT Filed: Nov. 29, 2006
(86) PCT No.: PCT/GB2007/004554

§ 371 Date: May 24, 2010

(30) Foreign Application Priority Data
Nov. 29, 2006 (GB) 0623833.1

(51) Int. Cl. A61K 38/45; C12Q 1/48; C12Q 1/68; G01N 33/50; A61K 31/7088; C07H 21/04; A61P 35/00

(52) U.S. Cl. 424/94.5; 435/15; 435/6; 435/7.1; 514/44 R; 536/23.2

(57) ABSTRACT

The present invention relates to the diagnosis, prognosis or predisposition of developing cancer, especially lymphoma. The invention also relates to gene therapy and identification of potential new therapeutic agents for treating cancer such as lymphoma.

### FIELD OF THE INVENTION

The present invention relates to the diagnosis, prognosis or predisposition of developing cancer, especially lymphoma. The invention also relates to gene therapy and identification of potential new therapeutic agents for treating cancer such as lymphoma.

### BACKGROUND TO THE INVENTION

The human genome encodes for 518 protein kinases, and approximately 70 of those are members of the serine/threonine AGC protein kinase A (PKA/PKG/PKG-like) class of protein kinases. PKA, PKG, PKB, p70 ribosomal S6 kinase (S6K), p90 ribosomal S6 kinase (PSK) and serum- and glucocorticoid-induced protein kinase (SGK) are only some of the members of this class. AGC kinases share structural similarities, and all the members of this class of protein kinases require phosphorylation of a conserved Ser/Thr residue within the activation segment for activation. AGC kinases have crucial roles in the regulation of physiological processes that are important for cell growth, metabolism, proliferation and survival.

The NDR (nuclear Dbf2-related) family of kinases represents a subclass of the AGC group of protein kinases¹. The human genome encodes four related kinases, NDR1 (also known as serine/threonine kinase 38 or STK38), NDR2 (or STK38L), LATS1 (large tumour suppressor-1) and LATS2. The NDR family is evolutionarily conserved and members can be found in Drosophila melanogaster (Trc(tricornered) and Lats/Warts), Caenorhabditis elegans (sensory axon guidance-1 (SAX-1) and LATS), Saccharomyces cerevisiae (Dbf2p, Dbf20p and Cbk1p), Schizosaccharomyces pombe (Sid2p and Orb6p), and some other fungi, protozoan and plant kinases.

NDR kinases have been shown to regulate mitosis, cell growth and development, and yeast studies have indicated the importance of the yeast NDR proteins for the life of a unicellular organism²⁻⁸. Recently, functions of metazoan NDR proteins in embryonic development, neurological processes and cancer biology have been suggested⁹⁻¹⁶.

Nonetheless, the physiological role for the NDR kinases NDR1 and NDR2 in mammals is not known. However, NDR1 cDNA can compensate for the loss of D. melanogaster Trc¹⁷ and NDR2 has a role in neuronal growth and differentiation¹⁸. However, some of the present inventors have recently reported that NDR1 is not crucial for mammalian development, as NDR1 deficient mice are viable and fertile. They also suggest that loss of NDR1 may be compensated for by the elevation of total NDR2 protein levels, as observed in specific tissues of NDR1 deficient mice e.g. thymus, lung a brain.

The present invention is based on further studies of the Ndr1^{-/-} mouse and the surprising observation that aged NDR1^{-/-} mice display a highly increased rate of high grade lymphoma.

### SUMMARY OF THE INVENTION

The present invention is based on observations by the present inventors that NRD1 knock-out mice although appearing viable, fertile and showing no apparent aberrant phenotype when young, when aged display a highly increased rate of developing high grade lymphoma.

Thus, in a first aspect there is provided a method of identifying subjects being predisposed or with an increased potential to suffering from a proliferative, neoplastic disease (e.g. a cancer), comprising the steps of:

detecting a level of NDR1 and/or NDR2 expression and/or activity in a sample from a test subject and identifying whether or not said level of expression and/or activity is decreased or upregulated with respect to a control or normal sample.

The sample may be isolated from a sample of tissue, or from a sample of body fluid, such as blood, mucous, urine or sputum. It may also be desirable to lyse cells within the sample, so as to release any NDR1 and/or NDR2 present. Lysis may be achieved by the use of solvents, such as SDS, sonication, mechanical disruption, a sudden drop in osmotic pressure and/or the use of one or more proteases to release some or all of any NDR1 and/or NDR2 present within the cells. The sample may be from any mammal, including sheep, cattle, dogs, cats, horses, etc., but is preferably from a human subject.

As mentioned, it is necessary to compare a level of NDR1 and/or NDR2 expression and/or activity with a control or “normal” sample, in order to detect whether or not a level of NDR1 and/or NDR2 expression and/or activity in the sample is increased or decreased in comparison to a control or normal sample. It is to be appreciated that the control or normal level may be a historical value, that is not determined at the same time as the test sample.

A control sample is understood to be a sample from a “normal” non diseased tissue and can therefore be considered as having a “normal” level of NRD1 and/or NDR2 expression and/or activity. Thus, the level found in a particular tissue from a subject, e.g. a sample of blood/tissue, may be compared with a control sample, e.g. a sample of normal tissue from a subject not suspected of being predisposed to developing the disease. Alternatively a population of subjects may be tested in order to obtain a distribution of values, from which a “normal” or typical level of NRD1 and/or NRD2 expression and/or activity can be predicted/postulated.

As well as identifying patients predisposed or with an increased probability of developing a cancer, the present invention may extend to testing cancer patients during cancer therapy, in order to observe if NDR1 and/or NDR2 levels or activity change in response to therapy.

Typically, it is expected that subjects displaying an aberhant level of expression and/or activity, will display a down-regulation in NDR1 expression/activity and/or an up-regulation in NDR2 expression/activity.

According to the method of the present invention, subjects suffering from such a proliferative disease can be screened in order to determine the expression and/or activity of NDR1 and/or NDR2. The method may be performed in vitro, e.g. on a sample of tumour tissue derived from the subject.

A level of expression of NDR1 and/or NDR2 and/or its activity may be assayed in the sample by any technical means on the basis of e.g. RNA expression using for example the technique of RT-PCR or on the basis of e.g. protein expression/modifications (e.g. phosphorylation) using for example the technique of Western blotting, immunocytochemistry, immunohistochemistry or immunoassays including ELISA, immunoprecipitation and electrophoresis assays. A patient's NDR1 and/or NDR2 gene sequence(s) may also be determined in order to identify one or more mutations which may affect expression and/or activity.

For example, ELISA (enzyme linked immunosorbent assay) type assays, immunoprecipitation type assays, conventional Western blotting assays, immunocytochemistry and immunohistochemistry assays using e.g. monoclonal or polyclonal antibodies may be utilized to determine levels of NDR1 and/or NDR2 protein and/or activity (e.g. phosphorylation).

An example of a typical immunoassay would comprise the step of exposing a sample, as defined hereinabove, to any antibody recognizing NDR1 and/or NDR2. This antibody may be either a polyclonal antibody which may be raised against purified NDR1 and/or NDR2 protein or phosphorylated NDR1 and/or NDR2 (or peptide sequences derived from the NDR1 and/or NDR2 protein sequence) according to techniques well known in the art (cf “Antibodies. A Laboratory Manual”, Harlow et al., 1988, Cold Spring Harbor Laboratory, NY-UA) or a monoclonal antibody which may be raised against purified NDR1 and/or NDR2 protein or phosphorylated NDR1 and/or NDR2 (or NDR1 and/or NDR2 derived peptides) or an immunogen preparation containing NDR1 and/or NDR2 and selected for its specificity and/or high affinity for NDR1 and/or NDR2 according to conventional techniques.

As will be recognized by those in the art, numerous types of immunoassays are available for use in the present invention. For instance homogeneous and heterogeneous assays, direct and indirect binding assays, competitive assays, and sandwich assays are well known in the art and described in numerous publications, e.g. “Antibodies. A Laboratory Manual”, Harlow et al., 1988, Cold Spring Harbor Laboratory, NY-USA.

The antibody recognizing NDR1 and/or NDR2 protein or active (e.g. phosphorylated) NDR1 and/or NDR2, or another component of the test kit may carry a label depending upon their application. A “label” means here a molecule which provides, directly or indirectly, a detectable signal. Various labels may be employed, such as radioisotopes (e.g. ¹²⁵I, ¹³¹I, technetium, indium, ³H and ¹⁴C), fluorescents, chemiluminescents, enzymes (e.g. peroxidise, alkaline phosphatise, β-D-galactosidase, glucose oxidase, glutamate decarboxylase and β-amylase), enzyme substrates, cofactors and inhibitors, particles (e.g. colloidal gold particles), combinations of ligands and receptor's (e.g. streptavidin and biotin) and the like. Enzymatic labels are advantageous because they allow a high sensitivity, comparable to that of radioactive labels, provide superior spatial resolution in a histological context, do not require particular safety precautions and can be used in commercially available automated systems. Enzymatic labels most widely used both in research as diagnostic applications are horseradish peroxidase and alkaline phosphatase.

Particularly useful immunoassays are sandwich assays and competitive inhibition immunoassays which use at least one monoclonal antibody as defined above.

The sandwich assay may be a homosandwich assay, a heterosandwich assay or a lectin-immunometric assay.

An alteration in activity of NDR1 and/or NDR2 may also be detected by way of a change in its phosphorylation status, which can easily be detected using antibodies which are specifically reactive to phosphorylated and non-phosphorylated forms, as well as the ability of NDR1 and/or NDR2 to be able to bind to its substrate, which again can be detected by the use of appropriate antibodies or using gel-shift assays known to the skilled addressee. Thus, it is understood that NDR1 and/or NDR2 activity can be extended to cover activity of the substrate/binding partner(s) for NDR1 and/or NDR2.

NDR1 and/or NDR2 expression may also be measured by two-dimensional (2-D) gel electrophoresis. 2-D gel electrophoresis is known in the art and typically involves isoelectric focusing (IEF) along a first dimension followed by SDS-PAGE (sodium dodecyl sulphate-polyacrylamide gel electrophoresis) along a second dimension. The resulting electropherograms are analyzed, for example, by immunoblot analysis using antibodies.

In a further aspect there is provided a method of identifying a potential therapeutic agent for use in treating, prophylatically or otherwise, a cancer, comprising the steps of:

a) providing a cell, tissue or animal which displays a down-regulation or upregulation of NDR1 and/or NDR2;

b) administering thereto a test therapeutic agent; and

c) observing whether or not the cell, tissue or animal displays an increase in cell proliferation or apoptosis.

The skilled addressee is well aware of tests known in the art to study cell proliferation and/or apoptosis. In the case of an animal, cell proliferation can be detected by growth of a tumour or cancer development, especially lymphoma development.

Typically the above method is conducted on a defined population of cells, tissue samples or animals which display an up or down-regulation of NDR1 and/or NDR2, in order that a statistically relevant distinction can be made between cells/tissues/animals which receive the test agent and those which do not. Thus, it is understood that not all samples treated with a particular test agent would display the same effect, but rather only a portion of a population may do so.

In instances where NDR1 and/or NDR2 is up-regulated, suitable test agents include kinase inhibitors known in the art. Examples of potentially suitable kinases may be found in, for example, D. Fabbro et al., Pharmacology & Therapeutics 93 (2992), p 79-98 and the papers referred to therein: WO06102079; U.S. Pat. No. 6,528,776.

In a further aspect, the present invention provides a method of identifying potential therapeutic targets for use in developing anti-cancer agents, the method comprising the steps of:

a) providing a cell, tissue or animal in which the expression and/or activity of NDR1 and/or NDR2 has been up or down-regulated; and

b) identifying therapeutic target genes which are differentially regulated as a consequence of NDR1 and/or NDR2 up or down-regulation.

NDR1 and/or NDR2 expression and/or activity can be up-regulated by the use of, for example, appropriate agonists, or by increasing the level of expression/activity by introduction of a recombinant molecule capable of expressing NDR1 and/or NDR2. Down-regulation of NDR1 and/or NDR2 can be achieved by generating knock-down or knock-out mutants, using techniques well known to the skilled addressee and described herein, or by way of RNAi technology again well known to the skilled addressee.

Once a target gene has been identified it may be used for drug screening purposes to seek to identify potential anti-cancer drugs. Such screens may involve the expression of the protein encoded by the target gene and chemical agents contacted with the protein in order to ascertain whether or not the chemical agent has any effect on the protein's activity.

Assays for protein activities of known function are know in the art. Generally such assays are termed functional assays and may be conducted in vitro in a cell free or cell based system. Where a functional assay is available, it is to be preferred to a ligand binding assay.

Assays for proteins of unknown function typically rely on assessment of ligand binding only, but other assays based on disturbance of chemical levels are well known to those of skill in the art.

The provision of candidate chemicals for use in the present invention are well known to those skilled in the art. For example libraries of compounds can be easily synthesised and tested. This is well described for example in: Applications of combinatorial technologies to drug discovery, 2. Combinatorial organic synthesis, library screening techniques, and future direction, J. Med. Chem., 1994, 37, 1385-1401.

For proteins of unknown function, the ligand binding assays outlined herein will also define a group of candidate chemicals. However, this group is likely to be large, since binding may occur to a number of different sites on the exposed surface of the protein, and binding alone does not predict the effect of ligand binding on the activity of the protein. Stringent selection among the candidate chemicals for those with the greatest affinity will define a set of chemicals small enough to be tested.

An alternative or additional procedure is to express the protein target in a cell which has been manipulated genetically to contain a sensor for calcium ions, cyclic-AMP or other components of cell signalling pathways. For example, permanent cell lines of any suitably origin may be transfected, and lines expressing the protein permanently selected. In many cases, expression of an unknown protein will cause a shift in the level of cell signalling components, which will be detected by the sensor and can be read, for example, as a fluorescent or luminescent signal. The difference between the protein-expressing cells and control cells forms the basis of the assay. Effects of chemicals on the difference between protein expressing and control lines are assessed.

Proteins for all the assays described can be produced by cloning the gene for example into plasmid vectors that allow high expression in a system of choice e.g. insect cell culture, yeast, animal cells, bacteria such as Escherichia coli. To enable effective purification of the protein, a vector may be used that incorporates an epitope tag (or other “sticky” extension such as His6) onto the protein on synthesis. A number of such vectors and purification systems are commercially available.

The polynucleotide fragment can be molecularly cloned into a prokaryotic or eukaryotic expression vector using standard techniques and administered to a host. The expression vector is taken up by cells and the polynucleotide fragment of interest expressed, producing protein.

The cloning and expression of a recombinant essential polynucleotide fragment also facilitates in producing antibodies and fragments thereof (particularly monoclonal antibodies).

It will be understood that for the particular polypeptides embraced herein, natural variations such as may occur due to polymorphisms, can exist between individuals or between members of the family. These variations may be demonstrated by (an) amino acid difference(s) in the overall sequence or by deletions, substitutions, insertions, inversions or additions of (an) amino acid(s) in said sequence. All such derivatives showing the recognised modulatory activity are included within the scope of the invention. For example, for the purpose of the present invention conservative replacements may be made between amino acids within the following groups:

(I) Alanine, serine, threonine;

(II) Glutamic acid and aspartic acid;

(III) Arginine and leucine;

(IV) Asparagine and glutamine;

(V) Isoleucine, leucine and valine;

(VI) Phenylalanine, tyrosine and tryptophan.

Moreover, recombinant DNA technology may be used to prepare nucleic acid sequences encoding the various derivatives outlined above.

As is well known in the art, the degeneracy of the genetic code permits substitution of bases in a codon resulting in a different codon which is still capable of coding for the same amino acid, e.g. the codon for amino acid glutamic acid is both GAT and GAA. Consequently, it is clear that for the expression of polypeptides from the genes described herein or fragments thereof, use can be made of a derivative nucleic acid sequence with such an alternative codon composition different from the gene sequences deposited in Genbank.

The polynucleotide fragments of the present invention are preferably linked to regulatory control sequences. Such control sequences may comprise promoters, operators, inducers, enhancers, silencers, ribosome binding sites, terminators etc. Suitable control sequences for a given host may be selected by those of ordinary skill in the art.

A polynucleotide fragment according to the present invention can be ligated to various expression controlling sequences, resulting in a so called recombinant nucleic acid molecule. Thus, the present invention also includes an expression vector containing an expressible nucleic acid molecule. The recombinant nucleic acid molecule can then be used for the transformation of a suitable host. Such hybrid molecules are preferably derived from, for example, plasmids or from nucleic acid sequences present in bacteriophages or viruses and are termed vector molecules.

Specific vectors which can be used to clone nucleic acid sequences according to the invention are known in the art (e.g. Rodriguez, E. L. and Denhadt, D. T., Edit., Vectors: a survey of molecular cloning vectors and their uses, Butterworths, 1998, or Jones et al., Vectors: Cloning Applications: Essential Techniques (Essential techniques series), John Wiley & Son. 1998).

The methods to be used for the construction of a recombinant nucleic acid molecule according to the invention are known to those of ordinary skill in the art and are inter alia set forth in Sambrook, et al. (Molecular Cloning: a laboratory manual Cold Spring Harbour Laboratory, 1989).

The present invention also relates to a transformed cell containing the polynucleotide fragment in an expressible form. “Transformation”, as used herein, refers to the introduction of a heterologous polynucleotide fragment into a host cell. The method used may be any known in the art, for example, direct uptake, transfection transduction or electroporation (Current Protocols in Molecular Biology, 1995. John Wiley and Sons Inc.). The heterologous polynucleotide fragment may be maintained through autonomous replication or alternatively, may be integrated into the host genome. The recombinant nucleic acid molecules preferably are provided with appropriate control sequences compatible with the designated host which can regulate the expression of the inserted polynucleotide fragment, e.g. tetracycline responsive promoter, thymidine kinase promoter, SV-40 promoter and the like.

Suitable hosts for the expression of recombinant nucleic acid molecules may be prokaryotic or eukaryotic in origin. Hosts suitable for the expression of recombinant nucleic acid molecules may be selected from bacteria, yeast, insect cells and mammalian cells.

### DEFINITIONS OF TERMS AS USED HEREIN

A “gene” as used herein is a nucleic acid molecule or polynucleotide composed of a discrete order of nucleotide bases. The term includes the ordering of bases that encodes a discrete product (i.e. “coding region”), whether RNA or proteinaceous in nature, as well as the ordered bases that precede or follow a “coding region”. Non-limiting examples of the latter include 5′ and 3′ untranslated regions of a gene. It is appreciated that more than one polynucleotide may be capable of encoding a discrete product. It is also appreciated that alleles and polymorphisms of the disclosed sequences may exist and may be used in the practice of the invention to identify the expression level(s) of the disclosed sequences or the allele or polymorphism. Identification of a sequence as an allele or polymorphism depends in part upon chromosomal location and ability to recombine during mitosis.

“Target gene(s)” as used herein, may be genes whose transcriptional or translational expression or activity is directly or indirectly modulated, i.e. increased or decreased by NDR1 and/or NDR2.

Up-regulation or down-regulation is understood to refer to an alteration in expression or activity of the NDR1 and/or NDR2 gene/protein typically in comparison to a normal range as determined for a population as a whole. The skilled addressee is well aware of how to determine what a normal range from a population of animals and consequently when a level of expression or activity is to be considered as up or down-regulated. For example, a level of expression or activity may be considered as up or down regulated if it displays, for example, greater than 5%, 10%, 20%, 30% or 50% of a difference from the normal range. Of course, if no expression or activity can be detected, then the NDR1 and/or NDR2 gene/protein can be considered as down-regulated.

In a further aspect the present invention provides use of an NDR1 and/or NDR2 gene or protein or active fragment thereof for the manufacture of a medicament for treating and/or preventing a cancer, such as a lymphoma, from developing in a subject displaying an up-regulation or down-regulation in expression and/or activity of NDR1 and/or NDR2.

The invention also extends to a method of treating a patient, such as a cancer patient, displaying an up or down-regulation of NDR1 and/or NDR2 expression or activity, comprising the step of administering to the patient an NDR1 and/or NDR2 gene, or protein or active fragment thereof.

It is to be understood that an active fragment is a fragment of the gene which is capable of generating an active protein fragment, or is an active protein fragment itself, which still displays NDR1 or NDR2 activity, such as kinase activity.

The therapy may simply be to redress a misbalance between NDR1 and NDR2 expression/activity, when one is up-regulated, or to increase expression/activity when one is down-regulated.

It will be appreciated that the present invention also therefore extends to methods of treating prophylactically or therapeutically a cancer such as a lymphoma by administering to a patient suffering, predisposed or predicted to be susceptible to developing such a cancer, a DNA construct comprising a NDR1 and/or NDR2 gene sequence or active fragment thereof, which gene sequence or fragment is capable of expressing one or more copies of the NDR1 and/or NDR2 protein or active fragment thereof in a subject, whereby expression of said one or more copies of said NDR1 and/or NDR2 protein serves to treat the subject, or minimise or reduce the likelihood of developing cancer.

Typically, the NDR1 and/or NDR2 gene sequence or fragment thereof may be administered to a subject in the form of a recombinant molecule comprising NDR1 and/or NDR2 gene sequence or active fragment under appropriate transcriptional/translational controls to allow expression of said NDR1 and/or NDR2 protein when administered to a subject. It will be appreciate that the NDR1 and/or NDR2 sequence or fragment may be under control of a suitable promoter, such as a constitutive and/or controllable promoter.

The present invention also therefore provides a recombinant molecule comprising an NDR1 and/or NDR2 gene sequence or active fragment thereof for use in therapy. The recombinant molecule may be in the form of a plasmid, phagemid or viral vector. Furthermore, recombinantly expressed, or chemically synthesised NDR1 and/or NDR2 protein, or functionally important fragments thereof, may be produced and applied to a subject in a suitable pharmaceutical vehicle, as a treatment or prophylactic measure for treating/preventing said aforementioned cancer.

Many different viral and non-viral vectors and methods of their delivery, for use in gene therapy, are known, such as adenovirus vectors, adeno-associated virus vectors, retrovirus vectors, lentiviral vectors, herpes virus vectors, liposomes, naked DNA administration and the like.

When it is appropriate to control NDR1 and/or NDR2 expression and/or activity which is up-regulated, this may be achieved by use of kinase inhibitors, protein fragments of NDR1 and/or NDR2 designed to competitively inhibit the up-regulated NDR1 and/or NDR2 activity, or by the use of RNAi techniques known in the art.

As will be understood, the present invention provides a correlation between NDR1 and/or NDR2 expression and/or activity and the development of a cancer, such as a lymphoma.

Thus, in a further aspect, the present invention provides a method of conducting a clinical trial for an anticancer drug, especially a drug for treating a lymphoma, comprising the steps of:

a) administering said drug to cancer cells/tissue or a subject suffering from a cancer; and

b) detecting whether or not a level and/or activity of NDR1 and/or NDR2 changes in response to administration of said drug.

Indeed, where a drug is shown to be effective in treating a cancer, such as lymphoma and this is correlated also with an effect on NDR1 and/or NDR2 expression and/or activity, patients receiving the drug can be screened for an alteration in NDR1 and/or NDR2 expression and/or activity in order to determine whether or not the treatment is likely to be effective in treating the cancer.

Preferably, the cancer which is the subject of the present invention, is a lymphoma, such as a high grade lymphoma.

Lymphoma is a variety of cancer that originates in lymphocytes. Collectively, lymphocytes form the specific cells of the reticuloendothelial system and circulate in the vessels of the lymphatic system. Just as there are many types of lymphocytes, so there are many types of lymphoma. Lymophomas are part of the broad group of diseases called haematological neoplasms.

The WHO Classification is the latest classification of lymphoma, published by the World Health Organization in 2001. It was based upon the “Revised European-American Lymphoma classification” (REAL).

This classification attempts to classify lymphomas by cell type, i.e. the normal cell type that most closely resembles the tumour. They are classified in three large groups: the B cell tumours, the T cell and natural killer cell tumours and Hodgkin lymphoma, and in addition there are a few other minor groups such as Immunodeficiency-Associated Lymphoproliferative Disorders and histiocytic tumors: (ICD-O codes are provided where available).

Mature B Cell Neoplasms include Chronic lymphocytic leukaemia/small lymphocytic lymphoma; B-cell prolymphocytic leukaemia; Lymphoplasmacytic lymphoma/Waldenstrom macroglobulinemia; Splenic marginal zone lymphoma; Plasma cell neoplasms; Plasma cell myeloma; plasmacytoma; Monoclonal immunoglobulin deposition diseases; Heavy chain diseases; Extranodal marginal zone B cell lymphoma (MALT lymphoma); Nodal marginal zone B cell lymphoma; Follicular lymphoma; Mantle cell lymphoma; Diffuse large B cell lymphoma; Mediastinal (thymic) large B cell lymphoma; Intravascular large B cell lymphoma; Primary effusion lymphoma; Burkitt lymphoma/leukaemia and lymphomatoid granulomatosis.

Mature T cell and Natural Killer (NK) Cell Neoplasms include T cell prolymphocytic leukaemia; T cell large granular lymphocytic leukaemia; Aggressive NK cell leukaemia; Adult T cell leukaemia/lymphoma; Extranodal NK/T cell lymphoma, nasal type; Enteropathy-type T cell lymphoma; Hepatosplenic T cell lymphoma; Blstic NK cell lymphoma; Mycosis fungoides/Sezary syndrome; Primary cutaneous CD30-positive T cell lymphoproliferative disorders; Primary cutaneous anaplastic large cell lymphoma; Lymphomatoid papulosis; Angioimmunoblastic T cell lymphoma; Peripheral T cell lymphoma, unspecified and Anaplastic large cell lymphoma.

Hodgkin Lymphoma include Nodular lymphocyte-predominant Hodgkin lymphoma; Classical Hodgkin lymphoma; Nodular sclerosis; Mixed cellularity; Lymphocyte-rich and Lymphocyte depleted.

Immunodeficiency-Associated Lymphoproliferative Disorders include Associated with a primary immune disorder; Associated with the Human Immunodeficiency Virus (HIV); Post-transplant and Associated with Methotrexate therapy.

Histiocytic and Dendritic Cell Neoplasms include Histiocytic sarcoma; Langerhans cell histiocytosis; Langerhans cell sarcoma; Interdigitating dendritic cell sarcoma/tumour; Follicular dendritic cell sarcoma/tumour and Dendritic cell sarcoma, unspecified.

All such lymphomas including histiocytic neoplasms are included within the present invention, with T-cell lymphomas being preferred.

The present inventors have observed that lymphoma development due to aberhant NDR1 and/or NDR2 expression is late onset, that is that it develops in older animals. As such early detection of deficiencies in NDR1 and/or NDR2 may allow for early treatment to occur, before any disease onset has occurred. It has also been observed that NDR1^{-/-} female animals are more prone to developing high grade lymphomas than male animals, increasing the importance of identifying aberhant NDR1 and/or NDR2 levels in female animals.

### DETAILED DESCRIPTION

The present invention will now be further described by way of example and with reference to the Figures which show:

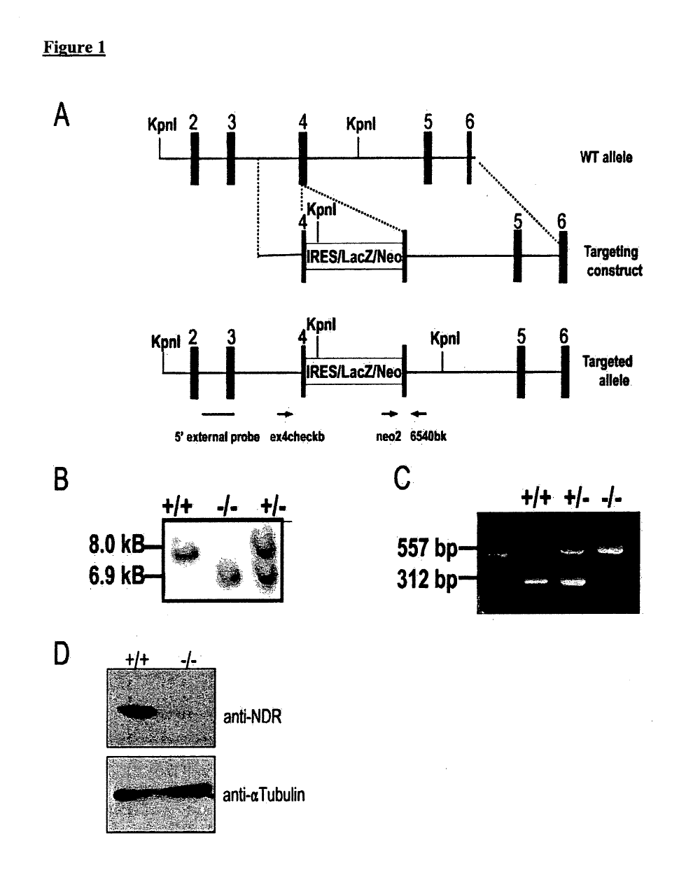

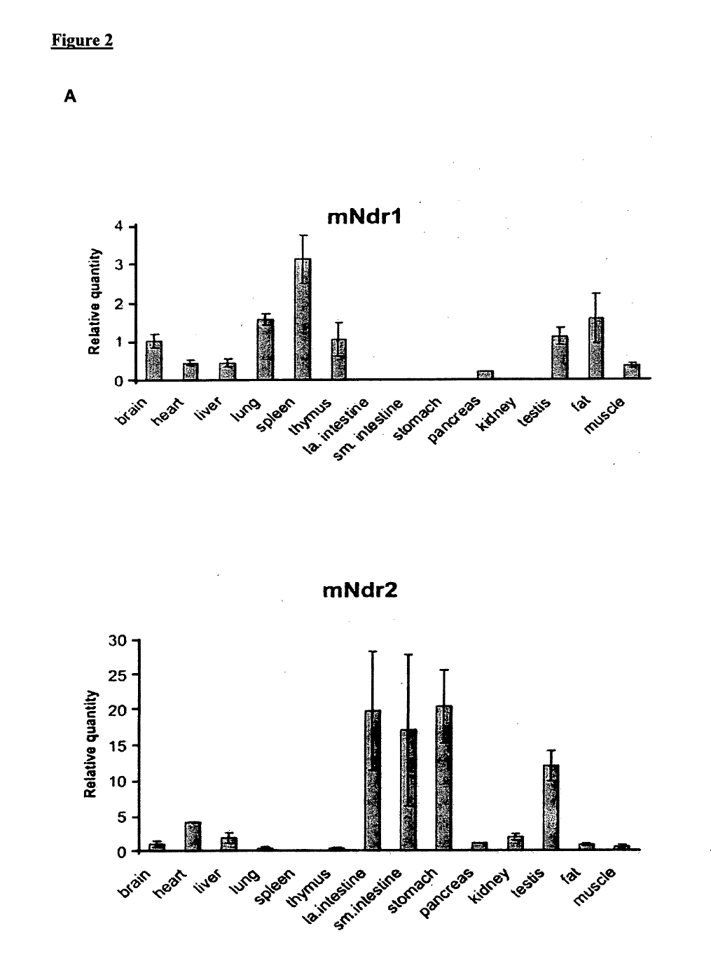

FIG. 1 shows

A: Diagram of the targeting strategy for generation NDR1^{-/-} mice. Schematic re-presentation of the NDR1 allele, targeting construct and targeted allele after re-combination. Arrows indicate the positions of the PCR primers used to genotype mice.
B: Southern blot of KpnI-digested DNA showing successful recombination. Binding site of the southern probe indicated in A.
C: Genotype PCR of tail biopsies.
D: Validation of the knockout by Western blot. In thymus samples of NDR1^{-/-} mice no NDR1 kinase could be detected;

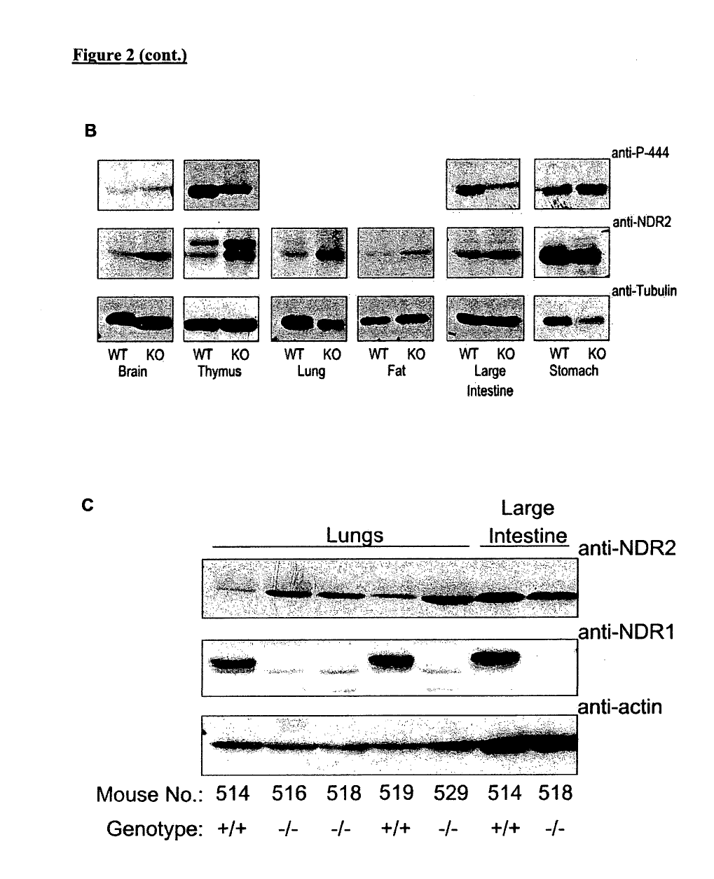

FIG. 2 shows the up-regulation of NDR 2 upon NDR1 loss in various mouse tissues:

A: Expression pattern of NDR1 and NDR2 in mice as determined by qPCR (from Stegert et al. JBC 2004)
B: NDR2 up-regulation in a variety of tissues from NDR1^{-/-} mice. Tissues showing high expression of NDR1 in wild-type mice show a strong increase in NDR2 level. Increase is weak in tissues showing no expression of NDR1.
C: Validation of findings in B with different mice.

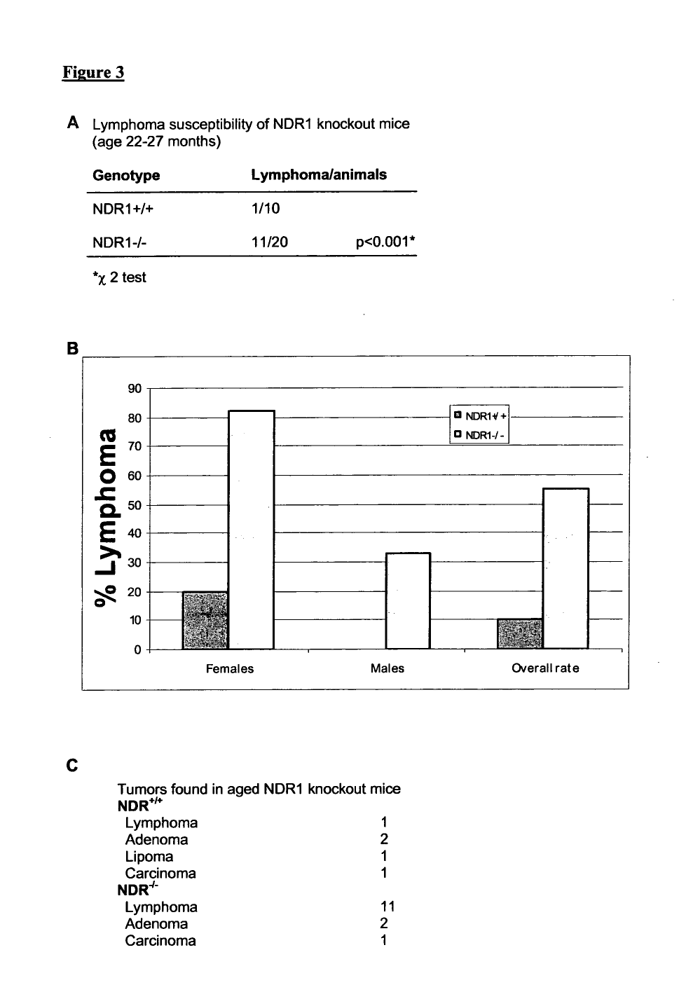

FIG. 3 shows increased lymphoma rate in aged NDR1^{-/-} mice,

A: Rate of high grade lymphoma in aged mice. Out of 20 analyzed NDR1^{-/-} animals 11 (55%) show lymphoma in various tissues.
B: Graphical representation of A. In addition the sex-specific rates of lymphoma incidents are included.
C: Tumor spectrum in aged NDR1^{-/-} and NDR1^{+/+} mice. The spectrum is not altered upon NDR1 deficiency.

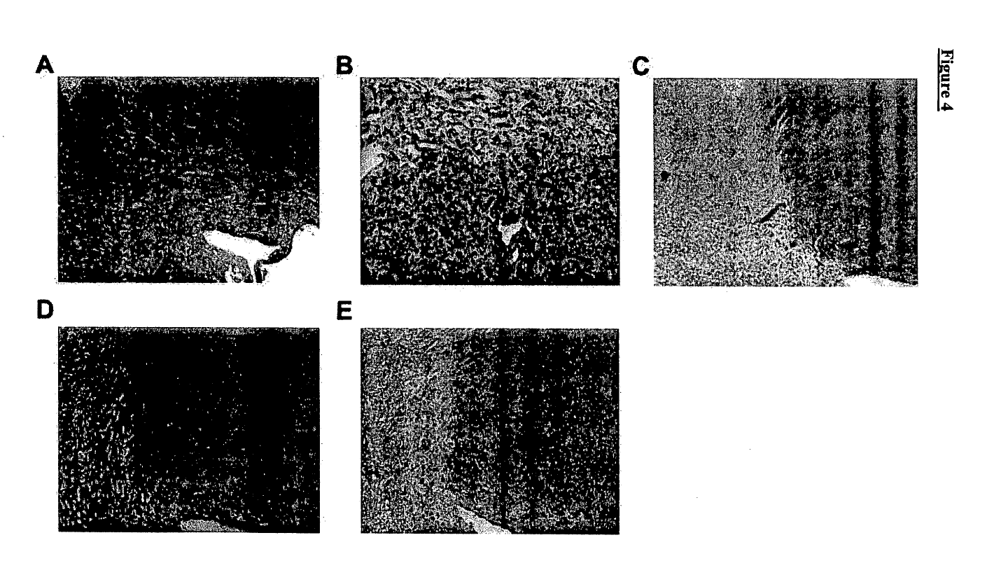

FIG. 4 shows immune-phenotyping and characterization of a representative lymphoma found in NDR1 deficient mice:

A: H&E staining of kidney tissue invaded by tumor cells (upper part; 10×)
B: Staining of the same tumor with pax-5, a pan B-cell marker, the staining of the tumor cells is negative (lower part; 10×)
C: Staining with CD3, a pan T-cell marker, tumor cells are positive (right; 10×)
D: Staining with CD4, tumor cells are negative (right; 10×)
E: Staining with CD8, tumor cells are positive (right, 10×)

### EXAMPLES SECTION

### Generation of Mice Deficient for NDR1

To investigate the biological function of murine NDR1, NDR1 deficient mice were generated by targeting exon 4 of the NDR1 gene, as shown in FIG. 1A. The targeted disruption of the NDR1 gene locus was confirmed by Southern blotting and PCR using tail-tissue samples from NDR1 wild type (NDR1^{+/+}), heterozygous (NDR1^{+/-}) and mutant (NDR1^{-/-}) mice (FIGS. 1B and C). On the protein level the absence of NDR1 was confirmed by using an antibody directed against the C-terminus of murine NDR1. In thymus samples from knockout mice no signal for NDR1 could be detected (FIG. 1D).

NDR1 knockout mice are viable, fertile and born in the expected Mendelian ratio (Table 1). As younger NDR1^{-/-} mice show no apparent developmental defects, 14-week-old mice (9-10) of each sex and genotype were subjected to a detailed analysis. Included were organ weight measurements of brain, heart, kidneys, liver, ovaries, spleen, testes and thymus and histological examination of adrenals, bone marrow, brain, caecum, colon, duodenum, gallbladder, heart, ileum, jejunum, kidneys, knee joint, liver, lung, auxiliary and mesenteric lymph nodes, ovaries, pancreas, peripheral nerve, pituitary, rectum, spleen, sternum, skeletal muscle, skin, spinal cord, sternum, stomach, testes, thymus and thyroid with parathyroid and trachea (data not shown).

TABLE 1
Ratio of NDR1 wild type, heterozygous and knockout animals
in comparison to the expected Mendelian ratios.

| Genotype | +/+ | −/+ | −/− |
|---|---|---|---|
| Number of animals | 63 | 119 | 60 |
| Obtained ratio | 26.03% | 49.17% | 24.79% |
| Expected ratio | 25% | 50% | 25% |

The performed weight analysis of NDR1^{-/-} and wildtype animals revealed a slight (but statistically not significant) decrease in the bodyweight of 6% in both sexes for the knockout animals. In addition a small weight decrease (14%) of the testis of male knockout mice was recorded. However, there were no clear correlating microscopic findings. No other organs and tissues of NDR1^{-/-} mice showed any major weight or histopathological differences when compared to wildtype mice.

There were also no significant differences between wild type and NDR1 deficient mice with regard to haematological evaluations, which included the analysis of complete blood cell counts as well as the analysis of hemoglobin and hematocrit values.

The lack of an apparent phenotype in younger NDR1^{-/-} mice could be explained by a compensation of NDR1 function by the other isoform NDR2. In tissue lysates from NDR1 KO mice a clear up-regulation of NDR2 was detected (FIG. 2B). This other isoform of NDR kinases shares 86% amino acid identity and so far no differences in biochemical regulation and activation between these two kinases have been reported. Interestingly, the observed increase in NDR2 levels seems to be tissue-specific as there is almost no change in the expression of NDR2 in tissues with low expression of NDR1 in wild-type.

### Aged Mice Deficient for NDR1 Show an Increased Rate of High-Grade Lymphoma

As younger mice show no apparent phenotype upon NDR1 deficiency, we also analyzed aged mice (23-26 months) to determine possible effects of NDR1-loss on the development of age related lesions. In an initial study we used 20 knockout and 10 wildtype animals of both sexes. Tissue sections of organs obtained from NDR1^{-/-} and NDR1^{+/+} mice were stained with H&E and subjected to histological analysis, which identified several tumors as well as two cases of hepatis peliosis in the knockout mice. Although the tumor spectrum is not altered in comparison to the wildtype, the incidence of lymphoma occurrence is strongly increased (FIG. 3).

In 55% of all NDR1^{-/-} animals high-grade lymphoma were identified, not only in lymphatic organs but also in other organs like liver, lung and kidney indicating a high rate of infiltration (FIG. 3B). Interestingly, female mice seem to be more prone to develop high grade lymphoma. In 82% of all female KO mice lymphoma were identified (FIG. 3B). We carried out immune phenotyping of the lesions and characterized them as being CD4−CD8+CD3+ high grade peripheral T-cell lymphoma (FIG. 4). Interestingly the only observed lymphoma in wild-type mice was identified as being CD4+CD8−CD3+, therefore a different kind of lesion (data not shown). This results clearly show an impact of NDR1 loss on the development of peripheral T-cell lymphoma. We found an up-regulation of NDR2 upon NDR1 ablation, interestingly Suzuki et al. could show a potential link between NDR2 up-regulation and the development of B-cell lymphoma. Future activities will address the question, whether NDR1 loss or NDR2 up-regulation is responsible for the observed increase in T-cell lymphoma rate.

### CONCLUSIONS AND OUTLOOK

NDR protein kinases are highly conserved from yeast to men, indicating an important function of this kinases. However it seems rather surprising, that mice deficient for NDR1 show at least in young animals a rather mild phenotype. Worms and flies only have one NDR kinase, SAX1 and TRICORNERED, and their ablation leads to more pronounced phenotypes. The organismal knockout of TRICORNERED is lethal. Due to an expansion of the kinome, mice and men have two NDR isoforms, therefore a functional compensation of NDR1 loss by NDR2 seems likely. Indeed we could show, that NDR2 protein levels are increased in mice lacking NDR1.

Aged mice, however, show a more pronounced phenotype with a strong increase in the rate of high grade lymphoma in NDR1 deficient mice. We observed in 55% of analyzed KO mice the occurrence of CD4−CD8+CD3+ peripheral T-cell lymphoma, indicating a role of NDR1 in the control of normal T-cell function and regulation. An impact of NDR1 on the immunological roles of T-cells seems likely.

### REFERENCES

1. Manning, G., Whyte D. B., Martinez, E., Hunter T. & Sundarsanam, S. The protein kinase complement of the human genome. Science 298, 1912-1934 (2002).
2. Colman-Lerner, A., Chin, T. E. & Brent, R. Yeast Cbk1 and Mob2 activate daughter-specific genetic programs to induce asymmetric cell fates. Cell 107, 739-750 (2001).
3. Du, L. L. & Novick, P. Pag 1p, a novel protein associated with protein kinase Cbk1p, is required for cell morphogenesis and proliferation in Saccharomyces cerevisiae. Mol. Biol. Cell 13, 503-514 (2002).
4. Hou, M. C., Guertin, D. A. 7 McCollum, D. Initiation of cytokinesis is controlled through multiple modes of regulation of the Sid2p-Mob1p kinase complex. Mol. Cell. Biol. 24, 3262-3276 (2004).
5. Hou, M. C., Salek, J. & McCollum, D. Mob1p interacts with the Sid2p kinase and is required for cytokinesis in fission yeast. Curr. Biol. 10, 619-622 (2000).
6. Hou, M. C., Wiley, D. J., Verde, F. & McCollum, D. Mob2p interacts with the protein kinase Orb6p to promote coordination of cell polarity with cell cycle progression. J. Cell Sci. 116, 125-135 (2003).
7. Komarnitsky, S. I. et al. DBF2 protein kinase binds to and acts through the cell cycle-regulated MOB1 protein. Mol. Cell. Biol. 18, 2100-2107 (1998).
8. Mah, A. S., Jang, J. & Deshaies, R. J. Protein kinase Cdc15 activates the Dbf2-Mob1 kinase complex. Proc. Natl. Acad. Sci. USA 98, 7325-7330 (2001).
9. Stoepel, J., Ottey, M. A., Kurischko, C., Hieter, P. & Luca, F. C. The miltotic exit network Mob1p-Dbf2p kinase complex localizes to the nucleus and regulates passenger protein localization. Mol. Biol. Cell 16, 5465-5479 (2005).
10. Weiss, E. L. et al. The Saccharomyces cerevisiae Mob2p-Cbk1p kinase complex promotes polarized growth and acts with the mitotic exit network to facilitate daughter cell-specific localization of Ace2p transcription factor. J. Cell Biol. 158, 885-900 (2002).
11. Emoto, K. et al. Control of dendritic branching and tiling by the Tricornered-kinase/Furry signalling pathway in Drosophila sensory neurons. Cell 119, 245-256 (2004).
12. Gallegos, M. E. & Bargmann, C. I. Mechanosensory neurite termination and tiling depend on SAX-2 and the SAX-1 kinase. Neuron 44, 239-249 (2004).
13. Harvey, K. F., Pfleger, C. M. & Hariharan, I. K. The Drosophila Mst orholog, hippo, restricts growth and cell proliferation and promotes apoptosis. Cell 114, 457-467 (2003).
14. He, Y. et al. Drosophila Mob family proteins interact with the related Tricornered (Trc) and Warts (Wts) kinases. Mol. Biol. Cell 16, 4139-4152 (2005).
15. He, Y., Fang, X., Emoto, K., Jan, Y. N. & Adler, P. N. The tricornered Ser/Thr protein kinase is regulated by phosphorylation and interacts with furry during Drosophila wing hair development. Mol. Biol. Cell 16, 689-700 (2005).
16. Huang, J., Wu, S., Barrera, J., Matthews, K. & Pan, D. The Hippo signalling pathway co-ordinately regulates cell proliferation and apoptosis by inactivating Yorkie, the Drosophila homolog of YAP. Cell 122, 421-434 (2005).
17. Jia., J. Zhang, W., Wang, D., Trinko, R. & Jiang, J. The Drosophila Step 20 family kinase dMST functions as a tumour suppressor by restricting cell proliferation and promoting apoptosis. Genes Dev. 17, 2514-2519 (2003).
18. Lai, Z. C. et al. Control of cell proliferation and apoptosis by Mob as tumour suppressor. Mats. Cell 120, 675-685 (2005).
19. Pantalacci, S., Tapon, N. & Leopold, P. The Salvador partner Hippo promotes apoptosis and cell-cycle exit in Drosophila. Nature Cell Biol. 5, 921-927 (2003).

1. A method of identifying subjects being predisposed or with an increased potential to suffering from a proliferative, neoplastic disease (e.g. a cancer), comprising:
detecting a level of NDR1 and/or NDR2 and/or substrate/binding partner expression and/or activity in a sample from a test subject and identifying whether or not said level of expression and/or activity is decreased or upregulated with respect to a control or normal sample.

2. A method of testing cancer patients during cancer therapy, in order to observe if NDR1 and/or NDR2 levels or activity change in response to therapy, comprising:
detecting a level of NDR1 and/or NDR2 and/or substrate/binding partner expression and/or activity in a sample from a subject undergoing cancer therapy, in order to detect whether or not said level or activity has changed in response to said therapy.

3. A method of screening cancer patients for assisting in determining an appropriate cancer therapy, comprising:
detecting whether or not a level of expression or activity of NDR1 and/or NDR2 and/or substrate/binding partner expression and/or activity in a sample from a subject displays an up or down-regulation in comparison to a normal or control sample.

4. The method according to claim 1 wherein the level of NDR1 expression and/or activity is down-regulated in the sample.

5. The method according to claim 1 wherein the level of NDR2 expression and/or activity is up-regulated in the sample.

6. The method according to claim 1 wherein the sample is a sample of tissue or body fluid.

7. The method according to claim 1 wherein the subject or patient is a human subject/patient.

8. The method according to claim 1 wherein the level of expression and/or activity is assayed on the basis of RNA expression, protein expression or protein modification.

9. The method according to claim 1 wherein the NDR1 and/or NDR2 gene sequence is determined in order to identify one or more mutations which can affect expression and/or activity.

10. The method according to claim 8 wherein a level of protein expression or modification is detected using a monoclonal or polyclonal antibody.

11. A method of identifying a potential therapeutic agent for use in treating, prophylatically or otherwise, a cancer, comprising:
a) providing a cell, tissue or animal which displays a down-regulation or upregulation of NDR1 and/or NDR2;
b) administering thereto a test therapeutic agent; and
c) observing whether or not the cell, tissue or animal displays altered cell proliferation or apoptosis.

12. A method of identifying potential therapeutic targets for use in developing anti-cancer agents, the method comprising:
a) providing a cell in which the expression and/or activity of NDR1 and/or NDR2 and/or substrate/binding partner has been up or down-regulated; and
b) identifying therapeutic target genes which are differentially regulated as a consequence of NDR1 and/or NDR2 and/or substrate/binding partner up or down-regulation.

13. A method for treating and/or preventing a cancer, such as a lymphoma, from developing in a subject displaying an up-regulation or down-regulation in expression and/or activity of NDR1 and/or NDR2, comprising administering an effective amount of NDR1 and/or NDR2 gene or protein or active fragment thereof.

14. A method of comprising administering a kinase inhibitor, protein fragment of NDR1 or NDR2, or RNAi molecule designed against NDR1 or NDR2 to a cancer patient.

15. A recombinant molecule comprising an NDR1 and/or NDR2 gene sequence or active fragment thereof for use in therapy.

16. The method or use according to claim 1 wherein the cancer is a lymphoma.

17. The method or use according to claim 16 wherein the lymphoma is a high grade lymphoma.

18. The method or use according to claim 16, wherein the lymphoma is a T-cell lymphoma.
